(19) United States
(12) Patent Application Publication
(10) Pub. No.: US 2006/0100583 A1
(43) Pub. Date: May 11, 2006

(54) Intra-venous (i.v.) blood catheter for subcutaneous infusion of liquids and/or drugs

(75) Inventor: Leonardo Terzoli, Rome (IT)

Correspondence Address: YOUNG & THOMPSON, 745 SOUTH 23RD STREET, 2ND FLOOR, ARLINGTON, VA, 22202, US

(73) Assignee: ANTEA ASSOCIAZIONE, ROME (IT)

(21) Appl. No.: 10/519,458

(22) PCT Filed: Feb. 19, 2002
(86) PCT No.: PCT/IT02/00097

§ 371 Date: Dec. 29, 2004

(51) Int. Cl. A61M 5/178

(52) U.S. Cl. 604/164.01; 604/264

(57) ABSTRACT

An intra-venous (I.V.) catheter used for the subcutaneous administration of liquids or drugs includes an introducer-needle (2) guiding a cannula or catheter made of flexible material. The catheter (6) has, in addition to the ejection-hole (16) at the distal end, several additional holes (8), placed all over the lateral surface of the catheter. This enables to obtain a homogeneous and continuous diffusion of the infused liquid on a larger area of the patient's subcutis, avoiding formation of pomphus.

The present invention relates to the field of medical-surgical devices and more particularly it concerns the improvement of intra-venous (I.V.) catheters used for subcutaneous administration of infusions and drugs.

Namely, an Intra-venous (I.V.) catheter is a device having an internal needle for the insertion of a catheter, consisting of an introducer-needle, usually made of steel, and an external cannula, preferably a teflon made tube having ultraslim walls that ensures the maximum flowability and flexibility and reduces trauma. After the tip of the needle, which protrudes from the distal end of the cannula, penetrates the patient's subcutaneous layer (under the derma), the operator removes the needle leaving the catheter in situ in order to connect said catheter to a syringe having no needle or any other infusion devices.

In the last years the technique of subcutaneously infusing liquids or drugs has became widespread probably because patients tolerate subcutaneous catheters better than endovenous catheters and, in addition, the use of subcutaneous catheters needs less precautions and they can easily used for domiciliary treatments.

However, traditional intra-venous (I.V.) catheters nowadays used, and commercially available, for subcutaneous administration of infusions and drugs are extremely sterilised, stable and reliable but they were conceived to be used but for a different utilisation: the endovenous administration.

People skilled in the field are aware that as above it can be a serious disadvantage, which can compromise the positive result of a therapy. We refer to the fact that no attention was paid on fluids dynamic, according to which the distal end of the catheter is the unique responsible for the downflow of administrated fluids.

Inevitably, when the catheter is subcutaneously inserted, real pomphus, macro-collection of liquid in localised areas of the patient's subcutis, appear and their size depends on the amount of the injected liquid.

This fact becomes really important in case of patients undergoing long-term administration, because frequently pomphus deriving from previous treatments can not be completely reabsorbed.

Object of the present invention is to overcome this disadvantage modifying traditional intra-venous (I.V.) catheters.

This was obtained, according to the invention, providing the catheter, in addition to the ejection-hole at the distal end, with supplementary holes, placed all over the catheter's surface.

Said holes ensure an homogeneous distribution of the injected liquid over a wider area of the subcutis, the extension of this area being directly proportional to the number of holes; in this manner formation of pomphus, as with traditional I.V. catheters, is avoided.

Further features and advantages of the invention will be more readily apparent from the following detailed description with reference to the accompanying drawings.

In the drawings:

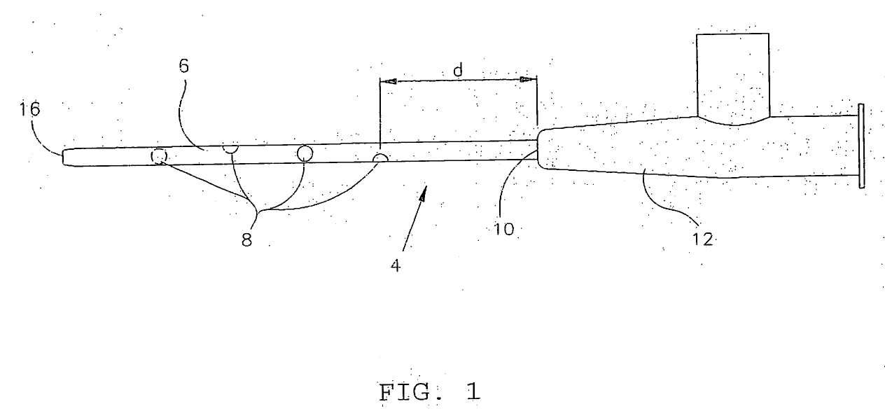

FIG. 1 shows the cannula having secondary holes placed all over the lateral surface;

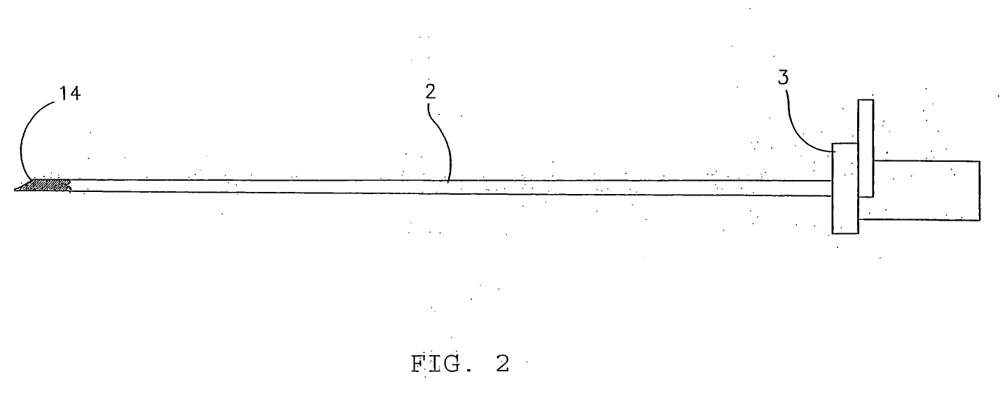

FIG. 2 shows the guide-needle, when it is drawn from the cannula;

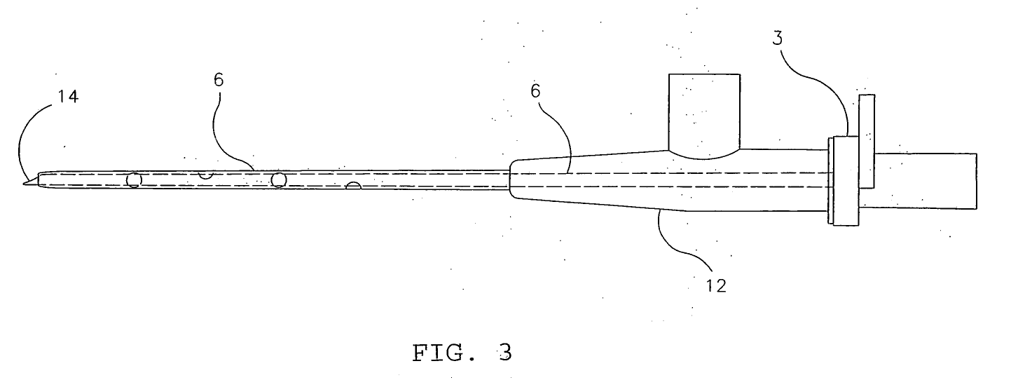

FIG. 3 shows the device of the present invention, ready to be use, with the needle inserted inside the cannula;

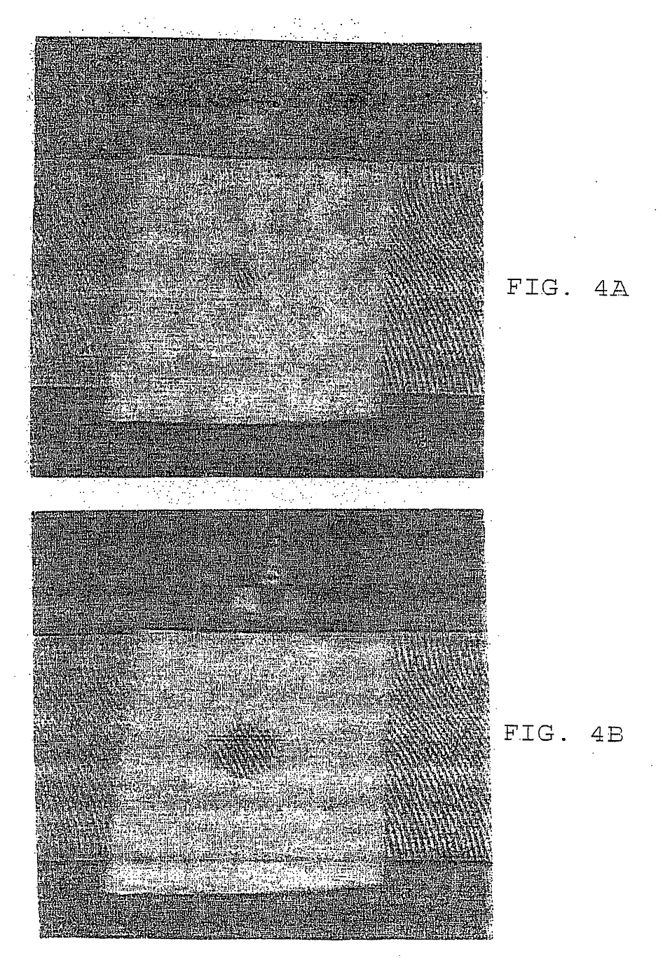

FIGS. 4a,4b and 5a,5b shows the results of a diffusion assay carried out on gauze where a liquid is infused using a traditional I.V. catheter and using the I.V. catheter of the present invention, at the time t₀ and at the time t₁ respectively;

With reference to the drawings, the I.V. catheter of the present invention, generically named as 4, does not differ, from commercially available I.V. catheters, as far as the materials used, the systems for the connection to the syringe and the systems for the infusion of the catheter, are concerned.

It substantially consists in an introducer-needle 2 having a sharp tip which ensures the maximum penetration index, said introducer-needle is inserted inside a cannula or catheter consisting of a small tube 6, preferably teflon-made, assembled on a usually plastic made (polypropylene or similar) support 12. For example, the length of the cannula is about 35-45 mm and its diameter is between 0,7 and 1,8 mm.

The innovative feature of the invention is that the catheter 6 is characterised in having, in addition to the main ejection-hole 16 at the distal end, several holes 8 placed all over the lateral surface.

Obviously, the distribution and the dimension of said holes 8 have to ensure the solidity of the cannula 6 and not compromise its mechanical resistance. Consequently, the holes are placed in order to result not aligned along the same generative line, but angularly spaced all over the lateral surface of the cannula.

As an example, the diameter of the holes is between 1,7 and 2,5 mm. It is important to underline that the section of said holes is usually lower than the section of the main ejection-hole 16.

To ensure a uniform outflow of the liquid, the section of the holes 8 increases toward the distal end.

The first hole 8 is appropriately made on the catheter at a distance “d” from the area 10 where said catheter 6 is keyed on the terminal cone 12 for the connection to the syringe (not represented), in order to avoid discharge of the infused liquid from the hole for the insertion of the needle in the skin (in a backward manner). In a preferred embodiment, in an I.V. catheter having a total length of 45 mm, said distance “d” is around 10 mm.

Following said indications, the infused liquid spreads on a wider area of the subcutis which is directly proportional to the number of holes.

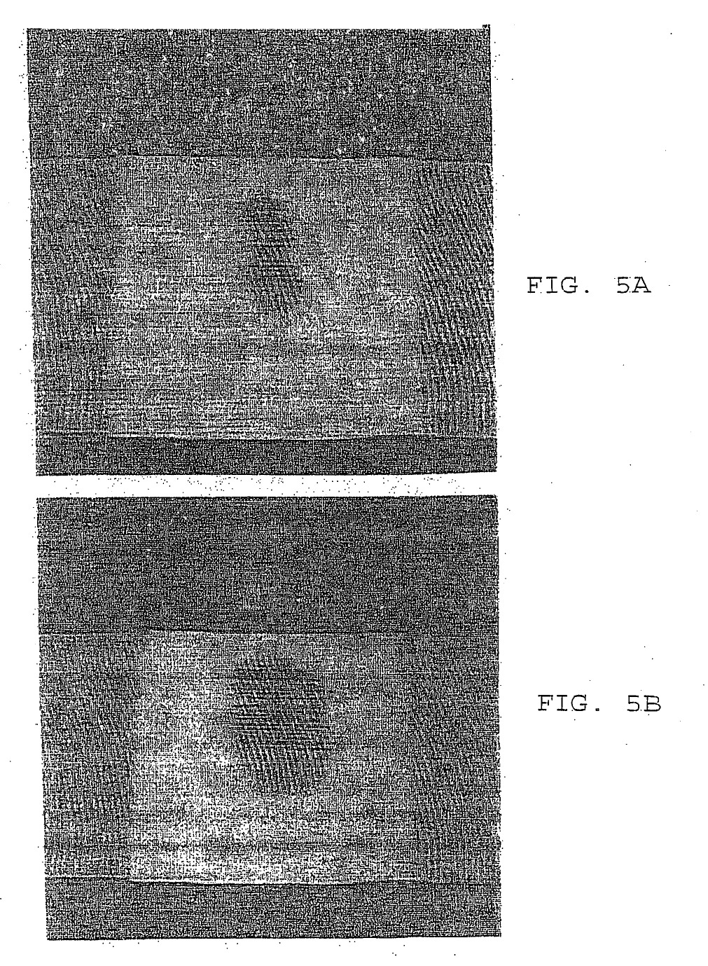

FIGS. 4a, 4b and 5a, 5b, show that no pomphus appear but the liquid spreads uniformly and homogeneously and does not concentrate in little areas.

Consequently, the liquid or the drug is more rapidly absorbed, the onset rate is quicker, the pharmacological effect is quickly reached and tissue unproper reactions and/or inflammation (phlogosis) are reduced.

It is important to underline that, using the device of the present invention, a lower hydrostatic pressure is exerted in the cannula and it ensures a reduced localised traumatism.

Moreover, as focused in FIG. 2, the device was specifically designed for a subcutaneous use and not for an endovenous use and for this reason it is not necessary that the guide-needle is perforated. This feature, has a key role in the prevention of diseases which might be transmitted by human fluids, as HIV, because it removes the risk related to the use of perforated needles. In fact, inside the cavity of perforated needles potentially infected residues deriving from tissue and/or fluids might remain, maintaining their infectivity because they do not come in contact with external air.

1. Intra-venous (I.V.) catheter for the subcutaneous administration of drugs comprising a introducer-needle (2) and a cannula (6) inserted on a support (12) to be connected to a syringe or other infusion devices, wherein said cannula (6) has in addition to the main ejection-hole (16) at the distal end, several additional holes (8), placed all over its lateral surface.

2. Intra-venous (I.V.) catheter according to claim 1 wherein said additional holes (8) are suitably spaced and unaligned in order to ensure a distribution of the infused product on a wider area of the subcutis directly proportional to the number of holes, and in order to avoid formation of pomphus resulting from the administration with traditional I.V. catheter.

3. Intra-venous (I.V.) catheter according to claim 1 wherein the first additional hole (8) is made at a distance (d) from the dovetail (10) of the cannula on the support (12) where the syringe or an other infusion device is inserted, and this distance is sufficient to avoid discharge of the infused liquid from the hole for the insertion of the needle in the skin, in a backward manner.

4. Intra-venous (I.V.) catheter according to claim 1 wherein the section of each additional hole (8) is smaller than the section of the main ejection-hole (16) at the distal end.

5. Intra-venous (I.V.) catheter according to claim 1 wherein the section of additional holes (8) increases toward the distal end of the cannula.

6. Intra-venous (I.V.) catheter according to claim 1 wherein the introducer-needle (2) is not perforated.

7. Intra-venous (I.V.) catheter according to claim 1 wherein the cannula (6) is made of plastic material opaque to radiations.

8. Intra-venous (I.V.) catheter according to claim 1 wherein the cannula (6) is made of transparent and flexible plastic material.
